(19) United States
(12) Patent Application Publication
(10) Pub. No.: US 2016/0089310 A1
(43) Pub. Date: Mar. 31, 2016

(54) TOPICAL COMPOSITIONS

(71) Applicant: DSM IP ASSETS B.V., Te Heerlen (NL)

(72) Inventors: Achim BROCK, Basel (CH); Christian GSTOETTMAYR, Basel (CH); Aline HUEBER, Basel (CH); Szilvia MESAROS, Basel (CH)

(21) Appl. No.: 14/890,759

(22) PCT Filed: May 14, 2014
(86) PCT No.: PCT/EP2014/059822

§ 371 Date: Nov. 12, 2015

(30) Foreign Application Priority Data
May 16, 2013 (EP) 13168068.8

(51) Int. Cl. A61K 8/02; A61Q 19/00; A61K 8/81

(52) U.S. Cl. CPC A61K 8/0279; A61K 8/8152; A61Q 19/00

(57) ABSTRACT

The present invention relates to a topical composition comprising at least one ingredient selected from the group consisting of a sebum control agent, a skin exfoliation agent and/or a collagen enhancing agent and a porous cross-linked polymethylmethacrylate bead having a particle size Dᵥ0 of greater 0.3 μm, a Dᵥ100 of less than 35 μm, a Dᵥ50 selected in the range of 6 to 15 μm and an oil absorption capacity selected in the range of 1.2 cc/g-2.5 cc/g.

The present invention relates to a topical composition comprising at least one ingredient selected from the group consisting of a sebum control agent, a skin exfoliation agent and/or a collagen enhancing agent and a porous cross-linked polymethylmethacrylate bead having a particle size Dᵥ0 of greater 0.3 μm, a Dᵥ100 of less than 35 μm, a Dᵥ50 selected in the range of 6 to 15 μm and an oil absorption capacity selected in the range of 1.2 cc/g-2.5 cc/g.

Human skin naturally produces and secrets an oily substance called sebum from sebaceous glands located near the skin surface. Sebum lubricates and protects skin against moisture loss by forming a film over the surface of the skin. A build-up of sebum on the surface of skin can cause the skin to appear shiny or oily. Besides the visually unappealing appearance of shiny or oily skin, sebum build-up can also highlight skin imperfections, promote skin acne development, and reduce the wearability of cosmetic compositions such as foundations. Thus, there is an ongoing need for topical compositions which instantly refine the skin appearance with controlled shine and pore minimization but also provide long term sebum control.

Furthermore, there is an ongoing need to further improve the complex sensory properties of topical compositions in order to fulfill the demanding expectations on the part of the end consumers which is economical and thus does not need high amounts of a texturing agent.

Surprisingly it has been found that the combination of a specific texturing agent and at least one ingredient selected from the group consisting of a sebum control agent, a skin exfoliation agent, and/or a collagen enhancing agent leads to a visibly refined skin that feels soft and smooth and furthermore reduces the sebum spot number and sebum production. Furthermore, such topical compositions exhibit an improved sensory profile and result in a velvet skin feel. Furthermore, the skin exhibits a matte finish imparted by the formulation.

Thus, the present invention relates to a topical composition comprising at least one ingredient selected from the group consisting of a sebum control agent, a skin exfoliation agent and/or a collagen enhancing agent, characterized in that the topical composition further comprises a porous cross-linked polymethylmethacrylate bead having a particle size Dᵥ0 of greater 0.3 μm, a Dᵥ100 of less than 35 μm, a Dᵥ50 selected in the range of 6 to 15 μm and an oil absorption capacity selected in the range of 1.2-2.5 cc/g in an amount selected in the range of 0.1 to 5 wt.-% based on the total weight of the composition.

Another subject matter of the invention is directed to the use of a porous polymethylmethacrylate bead having a particle size Dᵥ0 of greater 0.3 μm, a Dᵥ100 of less than 35 μm, a Dᵥ50 selected in the range of 6 to 15 μm and an oil absorption capacity in the range of 1.2-2.5 cc/g in an amount selected in the range of 0.1 to 5 wt.-% based on the total weight of the composition in combination with at least one ingredient selected from the group consisting of a sebum control agent, a skin exfoliation agent and/or a collagen enhancing agent to refine the short and long term skin appearance and/or to improve the sensory properties of a topical composition. Preferably, the topical compositions according to the present invention are used for short and long term gloss reduction, pore minimization and sebum prevention and reduction.

Furthermore, the invention is directed to a method for improving the short and long term skin appearance and/or the sensory properties of a topical composition said method comprising applying to the skin a topical composition comprising at least one ingredient selected from the group consisting of a sebum control agent, a skin exfoliation agent and/or a collagen enhancing agent, characterized in that the topical composition further comprises a porous cross-linked polymethylmethacrylate bead having a particle size Dᵥ0 of greater 0.3 μm, a Dᵥ100 of less than 35 μm, a Dᵥ50 selected in the range of 6 to 15 μm and an oil absorption capacity selected in the range of 1.2-2.5 cc/g in an amount selected in the range of 0.1 to 5 wt.-% based on the total weight of the composition and appreciating the effect. Preferably, the method is used for short and long term gloss reduction, pore minimization and sebum prevention and reduction.

Preferably, the improved sensory properties are a reduction of oiliness and greasiness as well as a maintained slipperiness resulting in an overall improved dry and velvet skin feel.

In all embodiments of the present invention preferably the oil absorption capacity is selected in the range of 1.5-2.0 cc/g.

The term oil absorption capacity refers to the weight of a specific oil absorbed by a material, determined by a specific method. It includes the oil absorption capacity of the dry particles existing between the inherent voids within and on the surface of the particles. The oil absorption capacity as referred to in the present invention is determined at 23° C. by weighting 2 g of the respective beads into a 20 ml beaker glass. Then, liquid paraffin (Paraffinum Perliquidum PH.EUR. CAS 8042-47-5) is added. After addition of 4 to 5 drops of paraffin to the powder, mixing is performed using a spatula, and addition of paraffin is continued until conglomerates of oil and powder have formed. From this point, the paraffin is added one drop at a time and the mixture is then triturated with the spatula. The addition of oil is stopped when the loose and dry powder completely disappears and a highly viscous white to transparent homogeneous gel is obtained. The oil absorption capacity (cc/g) is then calculated by the volume of paraffin used (in cc) per g of the respective beads.

The term sebum control agent as used herein refers to any bioactive which is able to reduce sebum production and sebum spot numbers. Preferably, the sebum control agent is selected from the group consisting Epilobium fleischeri extract, argania spinosa kernel oil, serenoa serrulata fruit extract, sesamum indicum (sesame) seed oil, beta-sitosterol, Vitamin B5 (pantothenic acid), Vitamin B6 (pyridoxine) as well as mixtures thereof. Most preferably the sebum control agent is an Epilobium fleischeri extract which is e.g. commercially available at DSM Nutritional Products under the tradename ALPAFLOR® ALP®-SEBUM.

The amount of the sebum control agent can easily be adjusted by a person skilled in the art. Preferably the amount of the sebum control agent is selected in the range of 0.1-10 wt.-%, more preferably in the range of 0.5 to 10 wt.-%, and most preferably in the range of 1 to 5 wt. % based on the total weight of the composition.

The term skin exfoliation agent as used herein refers to any exfoliation agent commonly used in cosmetic applications. Preferably, the skin exfoliation agent is selected from the group consisting of alpha-hydroxy acids (AHAs), beta-hydroxy acids (BHAs) or enzymes such as salicylic acid, citric acid, lactic acid, malic acid, glycolic acid, or fruit enzymes as well as mixtures thereof with amino acids and/or arginine. Such exfoliation agents are e.g. commercially available as AH-Care™ Amphoteric Hydroxy Complex (BASF).

The amount of skin exfoliation agent can easily be adjusted by a person skilled in the art. Preferably the amount of the skin exfoliation agent (as active) is selected in the range of 0.01-5 wt.-%, more preferably in the range of 0.05 to 2 wt.-%, and most preferably in the range of 0.1 to 1wt.-% based on the total weight of the composition.

The term a collagen enhancing agent refers to any active agent which stimulates collagen synthesis. Preferred collagen enhancing agent encompass Vitamin C (ascorbic acid) as well as derivatives thereof, polypeptides such as more preferably peptides with 2 to 5 amino acids or collagen synthesis stimulating plant extracts as well as mixtures thereof.

The amount of the collagen enhancing agent can easily be adjusted by a person skilled in the art. Preferably the amount of the collagen enhancing agent is selected in the range of 0.01-10 wt.-%, more preferably in the range of 0.1 to 5 wt.-%, and most preferably in the range of 0.5 to 3 wt.-% based on the total weight of the composition. It is understood that the given amounts are based on the respective product as commercially available and not to the actual active ingredient within the respective product form.

Particular suitable Vitamin C derivatives to be used in the topical compositions according to the present invention are sodium ascorbyl phosphate which is commercially available as Stay-C® 50 at DSM Nutritional Products Ltd.

Further particular suitable collagen enhancing agents to be used in the topical compositions according to the present invention are the peptides (INCI names) Palmitoyl Tripeptide-5 (e.g. commercially available under the tradename SYN®-COLL at DSM Nutritional Products Ltd), Palmitoyl Tetrapeptide-7 (e.g. commercially available as Matrixyl® 3000 at Sederma); Acetyl Tetrapeptide-9 (e.g. commercially available as Dermican™ LS 9745 at Laboratories Serobiologiques/Cognis).

Particularly suitable collagen synthesis stimulating plant extracts to be used in the topical compositions according to the present invention are Pisum Sativum Extract e.g. commercially available under the tradename Proteasyl® TP LS 8657, Proteasyl® LS 8951 or Proteasyl® LS 9818 at BASF, Vigna Aconitifolia Extract e.g. commercially available under the tradename Vit-A-Like™ LS 9793 or Vit-A-Like™ LS 9898 at BASF as well as Hibiscus abelmoschus seed extract e.g. commercially available as Linefactor™ at BASF.

In all embodiments of the present invention, the amount of the porous polymethylmethacrylate bead is preferably selected in the range of 0.5 to 4 wt.-%, such as in particular in the range of 1.5 to 3.5 wt.-% based on the total weight of the composition.

The particle size (in volume %) as given in the present invention is determined by a Coulter LS13320 or Malvern Mastersizer 2000 according to standard methods in the art.

The porous polymethylmethacrylate beads according to the present invention are preferably obtained by copolymerization of a monomer mixture consisting of methyl methacrylate and ethylene glycol dimethacrylate in the presence of a porogen according to known methods in the art and as e.g. outlined in KR 2006036614 which is enclosed herein by reference.

The term ‘consisting of’ as used according to the present invention means that the total amount of monomers ideally sum up to 100 wt.-%. It is however not excluded that small amount of impurities or additives may be present such as e.g. in amounts of less than 5 wt.-%, preferably less than 3 wt.-% which are e.g. introduced via the respective raw materials.

The porogen is preferably selected from the group consisting of toluene, n-hexanone, methylisobutyl ketone and isoamyl alcohol.

Initiators for polymerizing the monomers to provide the porous polymethylmethacrylate beads of the invention are those which are normally suitable for free-radical polymerization of acrylate monomers and which are oil-soluble and have low solubility in water such as e.g. organic peroxides, organic peroxyesters and organic azo initiators. The initiator is generally used in an amount of about 0.01 to 1 wt.-% based on the total monomer content.

Optionally, a water soluble inhibitor can be added to inhibit polymerization in the water phase in order to prevent the formation of too much polymer by emulsion and/or solution polymerization in the water phase, which can result in bead agglomeration or emulsion type polymerization. Suitable inhibitors include those selected from thiosulfates, thiocyanates, water soluble hydroquinones and nitrites. When used, the water soluble inhibitor can generally be added in an amount of from about 0.01 to about 1 parts by weight based on 100 parts total monomer content.

Furthermore, a water soluble or water dispersible polymeric stabilizer is needed to stabilize the suspension and in order to obtain stable beads. The stabilizer is preferably a water soluble or water dispersible polymer such as e.g. polyvinylpyrrolidone, polyvinylmethylether, polyethyleneimine, polyvinylalcohol, gelatin, starch, (m)ethylcellulose, carboxymethylcellulose, hydroxyethylcellulose, hydroxypropyl ellulose, poly(meth)acrylic acid and their sodium salts, and the like. The stabilizer is preferably used in an amount of about 0.001 to 10 wt.-%, more preferable in an amount of about 0.01 to 1 wt-% based on the total monomer content.

The monomers, free-radical initiator, and any optional materials can be mixed together in the prescribed ratio to form a premix. The stabilizer can be combined with water and then with the premix to form an oil in water suspension. The resulting suspension typically comprises from about 10 to about 50 weight percent monomer premix and from about 90 to about 50 weight percent water phase. Bead-type suspension polymerization in accordance with the present invention is typically a thermally initiated polymerization and is preferably carried out with agitation for about 2 to about 16 hours at a temperature between about 40° C. and 90° C. After isolation of the beads according to standard methods such as filtration or centrifugation the beads are preferably washed e.g. with water and/or ethanol and subjected to an extended drying, preferably at about 40-100° C. and more preferably at about 80-100° C. in order to further reduce the residual monomer content to an amount of below 250 ppm such as in particular below 100 ppm and most particular below 50 ppm. The drying can be performed by commonly known means to a person skilled in the art such as e.g. using a fluidized bed dryer or a conventional oven. The drying time can be easily adjusted by a person skilled in the art and is usually carried out over a period of 3 to 40 h such as about 8 to 20 h and in particular about 8 to 10 h.

In all embodiments of the present invention the porous polymethylmethacrylate beads are preferably prepared by suspension polymerisation of a monomer mixture consisting of 10-90 wt.-% methyl methacrylate and 10-90 wt.-% ethylene glycol dimethacrylate, with the proviso that the sum of monomers sums up to 100 wt.-%, in the presence of a porogen selected from toluene, n-hexanone, methylisobutyl ketone and isoamyl alcohol and a stabilizer selected from the group consisting of polyvinyl pyrrolidone, polyvinylmethylether, polyethyleneimine, poly(acrylicacid), polyvinylalcohol, vinyl acetate copolymer and ethyl cellulose.

Particularly suitable porous polymethylmethacrylate beads according to the present invention have a Dᵥ50 selected in the range of 9 to 12 μm and an oil absorption capacity selected in the range of 1.5-2.0 cc/g. Furthermore, it is preferred that the residual monomer content is less than 50 ppm (determined by Gas Chromatography). It is furthermore advantageous if the beads exhibit as 10% aqueous dispersion in distilled water a pH in the range of 5.0-9.0. It is furthermore preferred if the porous polymethylmethacrylate beads have a water content of less than 1.5 wt.-% (determined by Karl Fischer titration).

Suitable porous polymethylmethacrylate beads according to the present invention having a particle size Dᵥ0 of greater 0.3 μm, a Dᵥ100 of less than 35 μm, a Dᵥ50 selected in the range of 6 to 15 μm and an oil absorption capacity in the range of 1.2-2.5 cc/g are e.g. commercially available able as VALVANCE™ Touch 150 at DSM Nutritional Products Ltd.

The term “topical” is understood here to mean external application to keratinous substances, which are in particular the skin, scalp, eyelashes, eyebrows, nails, mucous membranes and hair.

Preferably, the topical compositions according to the present invention are applied to the skin.

As the compositions according to the invention are intended for topical application, they comprise a physiologically acceptable medium, that is to say a medium compatible with keratinous substances, such as the skin, mucous membranes, and keratinous fibers. In particular the physiologically acceptable medium is a cosmetically acceptable carrier.

The term cosmetically acceptable carrier refers to all carriers and/or excipients and/or diluents conventionally used in cosmetic compositions.

Preferred topical compositions according to the invention are skin care preparations or functional preparations.

Examples of skin care preparations are, in particular, light protective preparations (sunscreens), anti-ageing preparations, preparations for the treatment of photo-ageing, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), skin lightening preparations as well as BB and CC Creams.

Examples of functional preparations are cosmetic or pharmaceutical compositions containing active ingredients such as hormone preparations, vitamin preparations, vegetable extract preparations, anti-ageing preparations, and/or antimicrobial (antibacterial or antifungal) preparations without being limited thereto.

In a particular embodiment the topical compositions according to the invention are skin care preparations, such as (body) milks, lotions, foundations, creams, creamgels, serums, toners or gels.

The topical compositions according to the present invention may be in the form of a suspension or dispersion in solvents or fatty substances, or alternatively in the form of an emulsion or micro emulsion (in particular of oil-in-water (O/W) or water-in-oil (W/O) type, silicone-in-water (Si/W) or water-in-silicone (W/Si) type, PIT-emulsion, multiple emulsion (e.g. oil-in-water-in oil (O/W/O) or water-in-oil-in-water (W/O/W) type), pickering emulsion, hydrogel, hydrodisperion, alcoholic gel, lipogel, one- or multiphase solution or vesicular dispersion or other usual forms, which can also be applied by pens, as masks or as sprays.

In one embodiment, the topical compositions according to the present invention are advantageously in the form of an oil-in-water (O/W) emulsion comprising an oily phase dispersed in an aqueous phase in the presence of an O/W emulsifier. The preparation of such O/W emulsions is well known to a person skilled in the art.

If the topical composition according to the invention is an O/W or Si/W emulsion, then it contains advantageously at least one O/W- or Si/W-emulsifier selected from the list of PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate , PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, Isosteareth-20, Ceteareth-20, Oleth-20, Steareth-20, Steareth-21, Ceteth-20, Isoceteth-20, Laureth-23, Steareth-100, Glyceryl Stearate Citrate, Glyceryl Stearate SE (self-emulsifying), stearic acid, salts of stearic acid, polyglyceryl-3-methylglycosedistearate. Further suitable emulsifiers are phosphate esters and the salts thereof such as cetyl phosphate (Amphisol® A), diethanolamine cetyl phosphate (Amphisol®DEA), potassium cetyl phosphate (Amphisol® K), sodiumcetearylsulfat, sodium glyceryl oleate phosphate, hydrogenated vegetable glycerides phosphate and mixtures thereof. Further suitable emulsifiers are sorbitan oleate, sorbitan sesquioleate, sorbitan isostearate, sorbitan trioleate, Cetearyl Glucoside, Lauryl Glucoside, Decyl Glucoside, Sodium Stearoyl Glutamate, Sucrose Polystearate and Hydrated Polyisobuten. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof.

The at least one O/W and/ or Si/W emulsifier is preferably used in an amount of 0.5 to 10 wt.-%, in particular in the range of 0.5 to 6 wt.-% such as more in particular in the range of 0.5 to 5 wt.-% such as most in particular in the range of 1 to 4 wt.-%, based on the total weight of the composition.

Particular suitable O/W emulsifiers according to the present invention encompass phosphate esters emulsifier of formula (II)

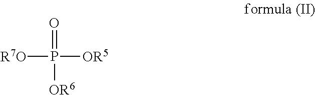

wherein R⁵, R⁶ and R⁷ may be hydrogen, an alkyl of from 1 to 22 carbons, preferably from 12 to 18 carbons; or an alkoxylated alkyl having 1 to 22 carbons, preferably from 12 to 18 carbons, and having 1 or more, preferably from 2 to 25, most preferably 2 to 12, moles ethylene oxide, with the provision that at least one of R⁵, R⁶ and R⁷ is an alkyl or alkoxylated alkyl as previously defined but having at least 6 alkyl carbons in said alkyl or alkoxylated alkyl group.

Monoesters in which R⁵ and R⁶ are hydrogen and R⁷ is selected from alkyl groups of 10 to 18 carbons and alkoxylated fatty alcohols of 10 to 18 carbons and 2 to 12 moles ethylene oxide are preferred. Among the preferred phosphate ester emulsifier are C₈₋₁₀ Alkyl Ethyl Phosphate, C₉₋₁₅ Alkyl Phosphate, Ceteareth-2 Phosphate, Ceteareth-5 Phosphate, Ceteth-8 Phosphate, Ceteth-10 Phosphate, Cetyl Phosphate, C6-10 Pareth-4 Phosphate, C₁₂-₁₅ Pareth-2 Phosphate, C₁₂₋₁₅ Pareth-3 Phosphate, DEA-Ceteareth-2 Phosphate, DEA-Cetyl Phosphate, DEA-Oleth-3 Phosphate, Potassium cetyl phosphate, Deceth-4 Phosphate, Deceth-6 Phosphate and Trilaureth-4 Phosphate. A particular advantageous phosphate ester emulsifier according to the invention is potassium cetyl phosphate e.g. commercially available as Amphisol® K at DSM Nutritional Products Ltd Kaiseraugst.

Further suitable O/W emulsifiers are polyethyleneglycol (PEG) esters or diesters such as e.g. [INCI Names] PEG-100 Stearate, PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate.

Particularly preferred according to the present invention is PEG-100 Stearate sold under the tradename Arlacel™ 165 (INCI Glyceryl Stearate (and) PEG-100 Stearate) by Croda.

Another particular suitable class of O/W emulsifiers are non-ionic self-emulsifying systems derived from olive oil e.g. known as (INCI Name) cetearyl olivate and sorbitan olivate (Chemical Composition: sorbitan ester and cetearyl ester of olive oil fatty acids) sold under the tradename OLIVEM 1000.

In particular embodiment, the invention relates to topical compositions in the form of O/W emulsions comprising an oily phase dispersed in an aqueous phase in the presence of an O/W emulsifier wherein the O/W emulsifier is selected from the group consisting of potassium cetyl phosphate, glyceryl stearate (and) PEG-100 Stearate, cetearyl olivate and sorbitan olivate as well as mixtures thereof.

In another preferred embodiment the topical compositions according to the present invention are hydrodispersions. Hydrodispersions are emulsifier-free preparations based on a liquid, semi-solid or solid internal (discontinuous) lipid phase in an external aqueous phase in the presence of a thickening agent.

Advantageous constituents of the lipid phase of the hydrodispersions according to the present invention encompass mineral oils, mineral waxes, liquid fatty acid triglycerides, natural oils, fats, waxes and other natural and synthetic fatty substances such as preferably esters of fatty acids with alcohols of low carbon number such as e.g. with isopropanol, propylene glycol or glycerol, or esters of fatty alcohols with alkanoic acids of low carbon number or with fatty acids as well as alkyl benzoates.

The lipid phase of the hydrodispersions according to the present invention is advantageously selected from esters of saturated and/or unsaturated, branched and/or unbranched alkanecarboxylic acids having a chain length of 3 to 30 carbon atoms and saturated and/or unsaturated, branched and/or unbranched alcohols having a chain length of 3 to 30 carbon atoms, or from esters of aromatic carboxylic acids and saturated and/or unsaturated, branched and/or unbranched alcohols having a chain length of 3 to 30 C atoms. Such esters are advantageously chosen from the group consisting of isopropyl myristate, isopropyl palmitate, isopropyl oleate, n-butyl stearate, n-hexyl laurate, n-decyl oleate, isononyl isononanoate, 2-ethylhexyl palmitate, 2-hexyl decyl stearate, oleyl oleate, oleyl erucyloleate, erucylerucate and synthetic, semi-synthetic and natural mixtures of such esters such as e.g. jojoba oil.

Furthermore, the lipid phase can advantageously be chosen from the group of branched and unbranched hydrocarbons and hydrocarbon waxes, silicone oils, dialkyl ethers or saturated or unsaturated, branched or unbranched alcohols and liquid fatty alcohol triglycerides. The liquid fatty alcohol triglycerides can advantageously be chosen from the group of synthetic, semi-synthetic and natural oils such as olive oil, sunflower oil, soy oil, peanut oil, almond oil, palm kernel oil, palm oil, rapeseed oil, coconut oil and the like more.

Advantageously, the lipid phase is selected from the group consisting of isohexadecane and/or cetearyl ethylhexanoate.

The amount of the lipid phase is preferably selected in the range of 5 to 30 wt.-%, more preferably in the range of 8-20 wt.-% such as most preferably in the range of 8 to 15 wt.-% based on the total weight of the hydrodispersion.

Suitable thickening agents are usual thickening agents employed in cosmetic compositions. Particularly suitable thickening agents according to the present invention are polyacrylic acid derivatives such as polyacrylates e.g. commercially available as Carbopoles, acrylic acid copolymers such as e.g. Pemulen TR1, Xanthan Gum or Ammonium Acryloyldimethyltaurate/VP crosspolymer such as Aristoflex AVC. Most preferred is the use of Ammonium Acryloyldimethyltaurate/VP crosspolymer.

The amount of the thickening agent in the hydrodispersion according to the present invention is preferably selected in the range of 0.05-5 wt -%, such as more preferably in the range of 0.1-2.5 wt -% based on the total weight of the hydrodispersion.

In a further preferred embodiment, the hydrodispersion in addition contains from 0.1 to 15 wt.-%, preferably from 0.5 to 10 wt.-%, most preferably from 1 to 7 wt.-% of glycerin.

The topical compositions according to the present invention furthermore advantageously contain at least one co-surfactant such as e.g. selected from the group of mono- and diglycerides and/or fatty alcohols. The co-surfactant is generally used in an amount selected in the range of 0.1 to 10 wt.-%, such as in particular in the range of 0.5 to 5 wt.-%, such as most in particular in the range of 1 to 3 wt.-%, based on the total weight of the composition. Particular suitable co-surfactants are selected from the list of alkyl alcohols such as cetyl alcohol (Lorol C16, Lanette 16) cetearyl alcohol (Lanette O), stearyl alcohol (Lanette 18), behenyl alcohol (Lanette 22), glyceryl stearate, glyceryl myristate (Estol 3650), hydrogenated coco-glycerides (Lipocire Na10) as well as mixtures thereof.

The compositions in form of O/W, Si/W, W/O or W/Si emulsions as well as hydrodispersions according to the invention can be provided, for example in the form of serum, milk or cream, and they are prepared according to the usual methods. The compositions which are subject-matters of the invention are intended for topical application and can in particular constitute a dermatological or cosmetic composition, for example intended for protecting human skin against the adverse effects of UV radiation (antiwrinkle, anti-ageing, moisturizing, anti-sun protection and the like).

According to an advantageous embodiment of the invention the compositions constitute cosmetic composition and are intended for topical application to the skin.

In accordance with the present invention, the topical compositions according to the invention may comprise further ingredients such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self-tanning, soothing, as well as agents to improve elasticity and skin barrier and/or further UV-filter substances and carriers and/or excipients or diluents conventionally used in topical compositions. If nothing else is stated, the excipients, additives, diluents, etc. mentioned in the following are suitable for topical compositions according to the present invention. The necessary amounts of the cosmetic and dermatological adjuvants and additives can, based on the desired product, easily be determined by the skilled person. The additional ingredients can either be added to the oily phase, the aqueous phase or separately as deemed appropriate. The mode of addition can easily be adapted by a person skilled in the art.

The cosmetically active ingredients useful herein can in some instances provide more than one benefit or operate via more than one mode of action.

The topical cosmetic compositions of the invention can also contain usual cosmetic adjuvants and additives, such as preservatives/antioxidants, fatty substances/oils, water, organic solvents, silicones, thickeners, softeners, emulsifiers, sunscreens, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, antifoaming agents, pigments or nanopigments, e.g. those suited for providing a photoprotective effect by physically blocking out ultraviolet radiation, or any other ingredients usually formulated into cosmetic compositions. Such cosmetic ingredients commonly used in the skin care industry, which are suitable for use in the compositions of the present invention are e.g. described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

The necessary amounts of the cosmetic and dermatological adjuvants and additives can—based on the desired product—easily be chosen by a skilled person in this field and will be illustrated in the examples, without being limited hereto.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions.

The topical compositions according to the invention in general have a pH in the range of 3 to 10, preferably a pH in the range of 4 to 8 and most preferably a pH in the range of 4 to 7.5. The pH can easily be adjusted as desired with suitable acids such as e.g. citric acid or bases such as sodium hydroxide (solution), Triethanolamine (TEA Care), Tromethamine (Trizma Base) and Aminomethyl Propanol (AMP-Ultra PC 2000) according to standard methods in the art.

The amount of the topical composition to be applied to the skin is not critical and can easily be adjusted by a person skilled in the art. Preferably the amount is selected in the range of 0.1 -3 mg/cm² skin, such as preferably in the range of 0.1 to 2 mg/cm² skin and most preferably in the range of 0.5 to 2 wt.-%/cm².

The following examples are provided to further illustrate the compositions and effects of the present invention. These examples are illustrative only and are not intended to limit the scope of the invention in any way.

### EXAMPLES

TABLE 1
Hydrodispersion

| | | | A | B |
|---|---|---|---|---|
| | Ingredient | INCI name | % w/w | % w/w |
| A | Arlamol HD | Isohexadecane | 8.0 | 8.0 |
| | Tegosoft liquid | Cetearyl Ethylhexanoate | 4.0 | 4.0 |
| | Aristoflex AVC | Ammonium | 1.0 | 1.0 |
| | | Acryloyldimethyltaurate/ | | |
| | | VP crosspolymer | | |
| B | Water dem. | Aqua | Ad 100 | Ad 100 |
| | Alpaflor ® | Glycerin, Aqua, citric acid, | 3.0 | 3.0 |
| | Alp ®-Sebum | potassium sorbate, | | |
| | | Epilobium fleischeri | | |
| | | extract | | |
| | Glycerin 99.5% | Glycerin | 3.0 | 3.0 |
| C | Euxyl PE 9010 | Phenoxyethanol, | q.s. | q.s. |
| | | Ethylhexylglycerin | | |
| D | Valvance ™ | Methyl Methacrylate | 3.0 | — |
| | Touch 150 | Cross Polymer | | |

Preparation:

1. Add phase B to A while stirring

2. Add C to AB and homogenize

3. Add phase D to the emulsion, and homogenize again

### Sensory Evaluation

The samples prepared as outlined above were tested in a blind study with a trained sensorial panel consisting of 6 persons under the following conditions: The evaluation takes part on the inner forearm; the panel leader applies 50 μL of the respective sample.

Evaluator spreads the product within a defined circle of 5 cm diameter using index or middle finger, circular motion, rate of 2 rotations/second. This is the so called rub-out phase. After the rub-out phase the immediate after-feel and/ or the 20 minutes after-feel was assessed according to standardized parameters. The intensities felt were quantified on a scale from 0 to 100 in comparison to training standards with known and defined sensory intensities.

TABLE 2
Results of the sensory evaluation - immediate after-feel

| | Oily | Greasy | Slipperiness |
|---|---|---|---|
| B (Reference) | 22 | 14.4 | 75.6 |
| A (Sample according to the invention) | 13.4 | 2.8 | 72.2 |
| Δ (value A-value B)* 100%/value B | −39% | −80% | −4.5% |

Oily: percentage of perceived oily character (liquid, fatty) of the residue
Greasy: percentage of perceived greasy character (solid, fatty) of the residue
Slipperiness: percentage of perceived fluidity after application of the residue

As can be retrieved from the results presented in table 2 the oily and greasy feeling is significantly reduced while the fluidity (parameter: slip) is maintained.

TABLE 3
Results of the gloss evaluation (blind study)

| | Immediate- | 20 min |
|---|---|---|
| | afterfeel | afterfeel |
| B (Reference) | 30.1 | 13.1 |
| A (Sample according to the invention) | 21.4 | 8.8 |
| Δ (value A-value B)* 100%/value B | −29% | −8.8% |

Gloss: amount of light reflected off skin (untreated skin = 10), absolute value (AV)

Furthermore, the gloss reduction was assessed on the face of one representative panelist in a half side test after 10 and 120 min versus non-treated (initial) and placebo treated skin by quantitative analysis of digital photographs of the face. The results are outline in table 4 and illustrate also a significant short and long term gloss reduction.

TABLE 4
Results of the gloss evaluation via digital photograph analysis

| Gloss reduction | Gloss reduction | Gloss reduction | Gloss reduction |
|---|---|---|---|
| vs. non treated | vs. placebo | vs. non treated | vs. placebo |
| 10 min after application | | 120 min after application | |
| −42% | −32% | −35% | −36% |

The results illustrate a strong and long lasting shine reduction.

### Sebum Reduction

Reduction of sebum spot and sebum production as detected by Sebutape after 56 days of treatment with 3 wt.-% of ALPAFLOR® ALP®-SEBUM versus Placebo without ALPAFLOR® ALP®-SEBUM. The results are presented in table 5.

TABLE 5
Result of sebum assessment

| Sebum spot number | Sebum production |
|---|---|
| −24% | −56% |

### Formulation Examples

Protecting Mattifying Cream

| Phase | Ingredients | INCI Name | % w/w |
|---|---|---|---|
| A | WATER DEM. | AQUA | ad 100 |
| | Dermofeel ® PA-3 | SODIUM PHYTATE, AQUA | 0.10 |
| | Glycerin 86.5% | GLYCERIN | 3.00 |
| | Preservatives | | q.s. |
| | Natriumhydroxid 10% solution | SODIUM HYDROXIDE, AQUA | 0.20 |
| B | Keltrol CG-T | XANTHAN GUM | 0.20 |
| | Veegum ®Ultra Granules | | 0.50 |
| C | Dermofeel ® GSC | GLYCERYL STEARATE CITRATE | 3.50 |
| | Tegosoft ® DC | DECYL COCOATE | 3.00 |
| | Jojoba Oil | SIMMONDSIA CHINENSIS SEED OIL | 2.00 |
| | Fitoderm ® | SQUALANE | 6.00 |
| | Lanette ® O | CETEARYL ALCOHOL | 2.00 |
| | Tegosoft ® CT | CAPRYLIC/CAPRIC TRIGLYCERIDE | 4.00 |
| D | DL-ALPHA-TOCOPHERYL | TOCOPHERYL ACETATE | 0.10 |
| | ACETATE | | |
| | ALPAFLOR ® ALP-SEBUM | GLYCERIN, AQUA, CITRIC ACID, | 3.00 |
| | | POTASSIUM SORBATE, EPILOBIUM | |
| | | FLEISCHERI EXTRACT | |
| E | VALVANCE ™ Touch 150 | Methyl Methacrylate Crosspolymer | 3.00 |

### Intensive Anti Wrinkle Treatment

| Phase | Ingredients | INCI Name | % w/w |
|---|---|---|---|
| A | WATER DEM. | AQUA | ad 100 |
| | Glycerin 86.5% | GLYCERIN | 5.00 |
| | SYN ®-COLL | PALMITOYL TRIPEPTIDE-5, GLYCERIN, | 2.50 |
| | | AQUA | |
| B | Crodafos CES | CETEARYL ALCOHOL, DICETYL | 5.00 |
| | | PHOSPHATE, CETETH-10 PHOSPHATE | |
| | Myritol 331 | COCOGLYCERIDES | 6.00 |
| | Tegosoft ® TN | C12-15 ALKYL BENZOATE | 3.00 |
| | Tegosoft ® DC | DECYL COCOATE | 3.00 |
| | Fitoderm ® | SQUALANE | 2.00 |
| C | Natriumhydroxid 10% | SODIUM HYDROXIDE, AQUA | q.s. |
| | Losung | | |
| D | Dow Corning 345 Fluid | CYCLOPENTASILOXAN (AND) | 3.00 |
| | | CYCLOHEXASILOXAN | |
| | Euxyl K 300 | PHENOXYETHANOL, METHYLPARABEN, | 0.80 |
| | | BUTYLPARABEN, ETHYLPARABEN, | |
| | | PROPYLPARABEN, ISOBUTYLPARABEN | |
| | Aristoflex AVC | AMMONIUM | 0.40 |
| | | ACRYLOYLDIMETHYLTAURATE/VP | |
| | | COPOLYMER | |
| F | Transcutol CG | ETHOXYDIGLYCOL | 5.00 |
| G | VALVANCE ™ Touch 150 | METHYL METHACRYLATE | 2.00 |
| | | CROSSPOLYMER | |

### Anti Wrinkle Eye Contour Cream

| | Ingredient | INCI name | % w/w |
|---|---|---|---|
| A | Amphisol ® K | Potassium Cetyl phosphate | 1.0 |
| | Lanette 16 | Cetyl Alcohol | 3.0 |
| | Cutina CP | Cetyl Palmitate | 1.5 |
| | Eutanol G | Octyldodecanol | 3.0 |
| | Cetiol SN | Cetearyl Isononanoate | 2.5 |
| | Pemulen TR-1 | Acrylate/C10-30 Alkyl Acrylate Crosspolymer | 0.10 |
| | VALVANCE ™ Touch 150 | METHYL METHACRYLATE CROSSPOLYMER | 2.0 |
| B | Glycerin 99.5% AMI | Glycerin PH. EUR. Vegetable | 3.0 |
| | Water dem. | Aqua ad | |
| | | 100 | |
| C | Regu ®-Age PF | Glycine Soja Protein, Hydrolyzed Rice Extract, | 3.0 |
| | | Superoxide Dismutase, Glycerin, Aqua, | |
| | | Phenoxyethanol, Sodium Benzoate, Potassium | |
| | | Sorbate, Sodium Dextran Sulfate | |
| | SYN ®-COLL | Palmitoyl Tripeptide-5, Glycerin, Aqua | 1.0 |
| | Euxyl PE 9010 | Phenoxyethanol, Ethylhexylglycerin | q.s. |

1. A topical composition comprising at least one ingredient selected from the group consisting of a sebum control agent, a skin exfoliation agent and/ or a collagen enhancing agent, wherein the topical composition further comprises a porous cross-linked polymethylmethacrylate bead having a particle size Dᵥ0 of greater 0.3 μm, a Dᵥ100 of less than 35 μm, a Dᵥ50 selected in the range of 6 to 15 μm and an oil absorption capacity selected in the range of 1.2-2.5 cc/g in an amount selected in the range of 0.1 to 5 wt.-% based on the total weight of the composition.

2. The topical composition according to claim 1, wherein the amount of the porous polymethylmethacrylate bead is selected in the range of 0.5-4 wt.-% based on the total weight of the cosmetic composition.

3. The topical composition according to claim 1, wherein the particles size Dᵥ50 is selected in the range of 9 to 12 μm and the oil absorption capacity is selected in the range of 1.5-2.0 cc/g.

4. The topical composition according to claim 1, wherein the sebum control agent is selected from the group consisting of Epilobium fleischeri extract, Argania spinosa kernel oil, Serenoa serrulata fruit extract, Sesamum indicum (sesame) seed oil, beta-sitosterol, Vitamin B5 (pantothenic acid), Vitamin B6 (pyridoxine) as well as mixtures thereof.

5. The topical composition according to claim 1, wherein the amount of the sebum control agent is selected in the range of 0.1-10 wt.-% based on the total weight of the composition.

6. The topical composition according to claim 1, wherein the skin exfoliation agent is selected from the group consisting of alpha-hydroxy acids (AHAs), beta-hydroxy acids (BHAs) or enzymes such as salicylic acid, citric acid, lactic acid, malic acid, glycolic acid, or fruit enzymes as well as mixtures thereof with amino acids and/or arginine.

7. The topical composition according to claim 1, wherein the amount of the skin exfoliation agent is selected in the range of 0.01-10 wt.-% based on the total weight of the composition.

8. The topical composition according to claim 1, wherein the collagen enhancing agent is selected from the group consisting of Vitamin C (ascorbic acid) as well as derivatives thereof, polypeptides such as more preferably peptides with 2 to 5 amino acids or collagen synthesis stimulating plant extracts as well as mixtures thereof.

9. The topical composition according to claim 1, wherein the amount of the collagen enhancing agent is selected in the range of 0.01-10 wt.-% based on the total weight of the composition.

10. The topical composition according to claim 1 wherein the porous polymethylmethacrylate bead is obtained by copolymerization of a monomer mixture consisting of methyl methacrylate and of ethylene glycol dimethacrylate in the presence of a porogen.

11. The topical composition according to claim 1, wherein the topical composition is a hydrodispersion.

12. Use of a porous polymethylmethacrylate bead having a particle size Dᵥ0 of greater 0.3 μm, a Dᵥ100 of less than 35 μm, a Dᵥ50 selected in the range of 6 to 15 μm and an oil absorption capacity in the range of 1.2-2.5 cc/g in an amount selected in the range of 0.1 to 5 wt.-% based on the total weight of the composition in combination with at least one ingredient selected from the group consisting of a sebum control agent, a skin exfoliation agent and/or a collagen enhancing agent to refine the short and long term skin appearance and/ or to improve the sensory properties of a cosmetic composition.

13. The use according to claim 12, wherein the refinement of the skin appearance is a short and long term gloss reduction, pore minimization and sebum prevention and reduction and the improved sensory properties are a reduction of oiliness and greasiness as well as a maintained slipperiness resulting in an overall improved dry and velvet skin feel.

14. A method for improving the short and long term skin appearance and/or the sensory properties of a topical composition said method comprising applying to the skin a topical composition comprising at least one ingredient selected from the group consisting of a sebum control agent, a skin exfoliation agent and/or a collagen enhancing agent, wherein the topical composition further comprises a porous cross-linked polymethylmethacrylate bead having a particle size Dᵥ0 of greater 0.3 μm, a Dᵥ100 of less than 35 μm, a Dᵥ50 selected in the range of 6 to 15 μm and an oil absorption capacity selected in the range of 1.2-2.5 cc/g in an amount selected in the range of 0.1 to 5 wt.-% based on the total weight of the composition and appreciating the effect.

15. The method according to claim 14, wherein the refinement of the skin appearance is a short and long term gloss reduction, pore minimization and sebum reduction and the improved sensory properties are an improved velvet skin feel.
